(19) United States
(12) Patent Application Publication
(10) Pub. No.: US 2006/0106449 A1
(43) Pub. Date: May 18, 2006

(54) Flow reducing implant

(75) Inventor: Shmuel Ben Muvhar, D.N. Beit Shean Valley (IL)

Correspondence Address: WOLF, BLOCK, SCHORR & SOLIS-COHEN LLP, 250 PARK AVENUE, NEW YORK, NY, 10177, US

(73) Assignee: Neovasc Medical Ltd., Or-Yehuda (IL)

(21) Appl. No.: 10/523,966

(22) PCT Filed: Aug. 7, 2003
(86) PCT No.: PCT/IL03/00659

§ 371 Date: Oct. 6, 2005

(30) Foreign Application Priority Data
Aug. 8, 2002 (IL) 151162
Oct. 3, 2002 (WO) PCT/IL02/00805
Oct. 17, 2002 (IL) 152366
Dec. 30, 2002 (IL) 153753
Apr. 10, 2003 (WO) PCT/IL03/00303

(51) Int. Cl. A61F 2/06; A61F 2/84; A61F 2/82

(52) U.S. Cl. 623/1.15; 606/194; 623/1.24

(57) ABSTRACT

An intra-vascular balloon (110), comprising a balloon body (1010); and at least one springy and elongate stave (1030) attached to said balloon and conforming to a surface of said balloon, such that said stave can apply contact force to an object in contact with said balloon.

### RELATED APPLICATIONS

This application is a continuation in part of U.S. application Ser. No. 09/534,968, filed Mar. 27, 2000 the disclosure of which is incorporated herein by reference. This application is also a continuation in part of PCT/IL01/00284, filed on Mar. 27, 2001 which designates the US and was published as PCT publication WO 01/72239 A2 in the English language. This application is also a continuation in part of PCT applications PCT/IL02/00805, published as WO 03/028522 and PCT/IL03/00303, filed Apr. 10, 2003. All of these PCT applications designate the US.

This application also claims priority from the following applications: Israel Application No. 151162, filed on Aug. 8, 2002, Israel Application No. 152366, filed on Oct. 17, 2002 and Israel Application No. 153753, filed on Dec. 30, 2002.

The disclosure of all of the above documents is incorporated herein by reference.

### FIELD OF THE INVENTION

The present invention relates to devices for reducing blood flow through the coronary sinus.

### BACKGROUND OF THE INVENTION

Occlusion of coronary arteries is a leading cause of death, especially sudden death, in what is commonly called a “heart attack”. When blood flow to a portion of the heart is suddenly stopped, the portion becomes ischemic and its electrical activity is disrupted. As the activity of the heart is mediated by electrical signal propagation, such disruption typically propagates to the rest of the heart, disorganizes the heart's activation and causes the heart output to be reduced drastically, which leads to ischemia and further damage beyond what was caused directly by the blockage.

If a patient survives the direct effects of the heart attack, the damage to the heart may predispose the patient to future electrical disorders and/or may significantly reduce the coronary output, thus reducing quality of life and life expectancy.

Angina pectoris is a chronic, or semi-chronic, ischemic coronary condition that occurs in the presence of occluded coronary arteries. Increased blood flow is required by the heart during exertion, but occluded arteries cannot provide the required increase in flow. The resultant ischemia produces pain, referred to as angina pectoris, that is not in itself life-threatening but may significantly reduce the quality of life.

The heart has natural mechanisms to overcome occlusion in coronary arteries. One such mechanism is angiogenesis, in which new arteries are created within the coronary tissue to bypass the occluded vessels. As angiogenesis does not usually occur to any great degree naturally, various procedures have been suggested to encourage it. For example Trans-Myocardial Revascularization (TMR) is a process in which multiple holes are drilled in the heart with the intent of causing new vessels to be created.

The venous circulation of the heart itself is primarily composed of a network of coronary veins that typically flow into a vein known as the coronary sinus. The coronary sinus is, “about 2 or 3 cm long, lying posterior in the coronary sulcus between the left atrium and ventricle. Its tributaries are the great, small and middle cardiac veins, the posterior vein of the left ventricle and the oblique vein of the left atrium, all except the last having valves at their orifices.” (Gray's Anatomy 38^{th} Edition, page 1575) The right atrium, into which the coronary sinus drains, collects all venous blood from the body.

Constriction of the coronary sinus to reduce the flow of venous blood that passes through it to the right atrium has been shown to promote angiogenesis. (“The Surgical Management of Coronary Artery Disease: Background, Rationale, Clinical Experience” by C. S. Beck and B. L. Brofman, 1956, by the American College of Physicians in Annals of Internal Medicine Vol. 45, No. 6, December 1956)

However, installing a coronary sinus constricting device requires open heart surgery and the temporary removal of the heart from the pericardium, a taxing procedure for any patient, particularly the patient with compromised coronary circulation. The method of promoting angiogenesis by installing a coronary sinus blood flow reducing implant during open-heart surgery, has fallen in disfavor, probably due to the hazardous associated installation procedure.

Ruiz in U.S. Pat. No. 6,120,534 teaches a flow reducing stent for use in a pulmonary artery to control damage to the lungs in a newborn that exhibits multiple, life-threatening cardio-pulmonary deformities. However, the thick, muscular, resilient walls of a pulmonary artery present a vastly different implant environment than the thin, weak non-muscular walls of the sinus and the flow dynamics that must be controlled in a pulmonary artery are vastly different than those of the coronary sinus.

U.S. application Ser. No. 09/534,968, filed Mar. 27, 2000 the disclosure of which is incorporated herein by reference, proposes a basic design for a coronary sinus flow restricting implant that is delivered percutaneously to its installation site and then expanded to provide flow reduction.

### Principles of Angiogeneis

To influence the flow of blood in a vessel of the body, there are many types of implants available, perhaps most notably, stents that expand within coronary arteries to increase blood flow along the vessel sector in which the stent is implanted. These flow-influencing implants differ from the present invention in a number of fundamental ways due to the radically divergent vessel architecture of the coronary sinus and/or the radically different goals for a flow reducing implant that is implanted in the coronary sinus.

The coronary sinus is a vein, albeit of a larger diameter than most veins, through which the blood from the various veins of the heart passes on its way to the right atrium from which it is sent to the lungs for oxygenation. The coronary sinus, like other veins of the body, lacks the thick muscular walls of arteries and may be damaged due to excess pressure. Hence, flow reducing implant should be transportable within blood vessels in a compact size and, following delivery, expand in the coronary sinus without causing undue stress on the relatively weak venous walls.

As the pressure the flow reducing implant places on the coronary sinus walls must be limited, additional methods may be required to anchor the flow reducing implant against the sinus walls. For example a flow reducing implant may promote coronary tissue ingrowth into its surface so it anchors properly in the coronary tissue.

Alternatively or additionally, the flow reducing implant should comprise materials that prevent coagulation, embolism formation and/or bacterial colonization in the coronary sinus and/or general circulation. Further, as the coronary sinus often exhibits varying cross sectional diameter and/or configuration along its length, the flow reducing implant may need to exhibit diameter variations that conform to the variable diameter of the coronary sinus.

There may be a fine line between the amount of reduction of blood flow that promotes angiogenesis and when such reduction causes untoward sequella, for example damage to coronary venous valves. Further, the amount of restriction in blood flow that is required to promote angiogenesis may vary from individual to individual and may not be readily apparent until following installation. Therefore, the flow reducing implant may require that the amount of flow reduction be adjustable in situ, perhaps even on multiple occasions, with low risk to the patient health.

Alternative or additional factors that promote angiogenesis may include changes in sinus blood flow dynamics. The flow reducing implant, therefore, may incorporate one or more design configurations to promote one or more changes in blood flow dynamics:

(a) Increased pressure in the coronary capillaries and/or increased perfusion duration.

(b) Increased resistance of the venous system to promote one or more of the following:
i) redistribution of blood flow in coronary arteries;
ii) increased intra-myocardial perfusion pressure; and
iii) increased intra-myocardial pressure.

(c) Increased arterial diastolic pressure (by restricting venous drainage) that causes the arterial auto-regulation to start working again, for example, such an auto regulation as described in Braunwald “Heart Disease: A Textbook of Cardiovascular Medicine”, 5th Edition, 1997, W. B. Saunders Company, Chapter 36, pages 1168-1169.

(d) Changes in pressure of sinus blood flow against the valve leading to the right atrium.

(e) Changes in blood stream dynamics such as laminar blood flow and/or blood stream rotation.

The amount of blood flow dynamics that stimulate angiogenesis may vary from individual to individual so the flow reducing implant may require a design that allows variation of blood flow dynamics, without risk to the patient, following implantation.

In an exemplary embodiment of the present invention, one or more of flow reducing implant designs may foster angiogenesis when implanted in one or more coronary arteries. Further, in an exemplary embodiment of the present invention, one or more features of flow reducing implant designs presented herein may foster angiogenesis when implanted in one or more coronary arteries. It is therefore understood that in accordance with promoting angiogenesis in the heart, any features of the flow reducing implants described herein may be modified for use in one or more coronary arteries.

In an exemplary embodiment of the present invention, one or more flow reducing implant designs may foster angiogenesis through implantation in one or more vessels of the body outside of the coronary vessels. For example, angiogenesis in the kidney may be promoted by implanting a flow reducing implant in a vessel of the kidney. It is therefore understood that in accordance with promoting angiogenesis in other regions of the body, the flow reducing implant described herein may be modified for use in one or more non-coronary vessels of the body.

### SUMMARY OF THE INVENTION

An aspect of some embodiments of the invention relates to a percutaneously deliverable flow reducing implant that reduces blood flow in the coronary sinus. In an exemplary embodiment of the present invention, the flow reducing implant promotes angiogenesis, thereby reducing ischemia and/or its crippling sequella including heart attack and death.

In an exemplary embodiment, the flow reducing implant comprises a hollow member having a flow passage in which at least a portion of said flow passage has a smaller cross section than a cross section of the coronary sinus. Optionally, the flow reducing implant is deliverable, for example, in a compact form via a delivery sheath to the coronary sinus where it attains its final configuration.

Optionally, the flow reducing implant configuration may be altered after implantation in the coronary sinus to change the amount of blood flow reduction and/or blood flow dynamics. Alternatively or additionally, the contact pressure between the flow reducing implant and the coronary sinus may be varied.

In an exemplary embodiment, at least a portion the flow reducing implant is self expanding. Optionally, the flow reducing implant comprises, for example, longitudinal and/or transverse slits of varying length to govern the expanded shape of the flow reducing implant. Optionally, the flow reducing implant comprises materials with a shape memory so the flow reducing implant automatically attains a desired shape following release, for example, from a delivery catheter into the coronary sinus. Alternatively or additionally, a standard catheter balloon is used to expand the flow reducing implant into its desired shape. Alternatively or additionally, a catheter balloon with a specialized shape is used to cause expansion of the flow reducing implant. Alternatively or additionally, the flow reducing implant is inflatable.

In an exemplary embodiment, the flow reducing implant comprises a material that changes size and/or configuration as it absorbs material from its environment. In an exemplary embodiment, the flow reducing implant absorbs liquid from the blood flowing through the coronary sinus to change its size and/or configuration.

In an exemplary embodiment, the flow reducing implant defines a flow passage that promotes angiogenesis by changing blood flow dynamics. For example, the flow reducing implant comprises at least one extension flap along its flow passage that extends, for example, into the flow passage. Optionally, the angle of the one or more flaps in relation to the blood flow, and/or its size, is adjustable following implantation of the flow reducing implant.

In an exemplary embodiment, the at least one extension extends from a sheath encircling at least a portion of the outside of the flow reducing implant. Optionally, the at least one extension comprises one or more curved members substantially planar with, for example, an outer surface of said flow reducing implant.

In an exemplary embodiment, the body of the flow reducing implant and/or the one or more extension flaps comprise a single solid wall. Alternatively or additionally, one or more extension flaps comprise outer and inner walls with a space between them. Optionally, the space is inflatable. Optionally, the flow reducing implant comprises two or more extension flaps, for example with one or more extension flaps located at each end of the flow reducing implant.

An aspect of some embodiments of the invention relates to a percutaneously deliverable coronary sinus flow reducing implant comprising at least one wire extending from at least one end of said flow reducing implant. In an exemplary embodiment, the at least one wire extends into the coronary sinus and is shaped to change blood flow dynamics, enhance anchoring of the flow reducing implant, and/or enhance reduction in size and/or positioning of the flow reducing implant. Alternatively or additionally, the at least one wire is attached along the flow passage and extends, for example, into the coronary sinus.

In an exemplary embodiment, the at least one wire comprises at least two wires. Optionally, the at least two wires are joined along their middle section within the flow passage of the flow reducing implant. Alternatively or additionally, the area where they are joined extends beyond the flow passage into the coronary sinus.

Alternatively or additionally, at least a portion of the central portions of said at least two wires are joined to a ring that alters blood flow dynamics. In an exemplary embodiment, the wires are joined to the ring in a manner that reduces blood turbulence, for example with curved connecting pieces to the ring. Alternatively or additionally, at least a portion of the central portions of said at least two wires are joined to a sphere, said sphere causing a change in blood flow dynamics to promote angiogenesis.

An aspect of some embodiments of the invention relates to a percutaneously deliverable coronary sinus flow reducing implant comprising at least one shape-conforming element that changes in geometry, thereby adjusting the size and/or configuration of the flow passage of the flow reducing implant. In an exemplary embodiment, the configuration of the one or more shape-conforming elements is governed by one or more impulses, for example, RF, ultrasound, low frequency sound, heat, electricity, electromagnetic and/or radiation. Optionally, one or more impulses are initiated from an initiation area near the one or more shape-conforming elements. Alternatively or additionally, the one or more impulses are initiated external to the heart, for example, external to the patient. In an exemplary embodiment, the configuration of the one or more shape-conforming elements is governed by one or more chemical reagents.

An aspect of some embodiments of the invention relates to a percutaneously deliverable coronary sinus flow reducing implant with one or more slits governing its implanted configuration and/or size and/or at least one ripple and/or a bend that defines and/or provides adjustment in configuration and/or size of the flow reducing implant.

In an exemplary embodiment, a flow reducing implant comprises a cord that, for example, encircles at least a portion of its diameter, in accordance with an exemplary embodiment of the invention. In an exemplary embodiment, the cord coronary sinus flow reducing implant changes in size and/or configuration by adjusting the size of the encircling cord and/or severing the cord.

Alternatively or additionally, the flow reducing implant wall has two edges that overlap each other. As the cord expands, the edges on the at least one wall of the cord type flow reducing implant move in relation to each other, thereby providing one or more expansion diameters.

An aspect of some embodiments of the invention relates to a percutaneously deliverable balloon catheter that achieves one or more expansion pressures to cause the expansion and/or modification of a flow reducing implant shape. In an exemplary embodiment, the balloon catheter comprises at least one stave along its surface that contacts at least a portion of a flow reducing implant during expansion of the flow reducing implant. In an exemplary embodiment, the balloon and/or one or more staves comprise materials configured to reduce in size to a compact profile, thereby allowing the catheter to be easily positioned and/or repositioned in relation to a flow reducing implant.

In an exemplary embodiment, the one or more staves of the balloon catheter comprise two or more staves. Optionally, the two or more staves are curved and/or connected at one or more places to provide a springy frame around the balloon. Optionally, the two or more curved staves foster, for example, a compact size during position and/or repositioning. Optionally, the balloon catheter comprises an inflatable design and thereby provides one or more expansion pressures in addition to the expansion pressure provided by the two or more springy staves, for implantation and/or positional adjustments of a flow reducing implant.

There is thus provided in accordance with an exemplary embodiment of the invention, a an intra-vascular balloon, comprising:

a balloon body; and

at least one springy and elongate stave attached to said balloon and conforming to a surface of said balloon, such that said stave can apply contact force to an object in contact with said balloon. Optionally, said balloon is elongate and wherein said stave is provided along a long dimension of said balloon. In an exemplary embodiment of the invention, said balloon comprises a tether attached to said balloon.

In an exemplary embodiment of the invention, said at least one stave comprise a plurality of staves arranged around said balloon. Optionally, said plurality of staves are attached to each other at their ends. Optionally, said staves modify a geometry of said balloon when not inflated. Optionally, said staves are configured to compact said balloon in a resting condition thereof. Alternatively, said staves are configured to apply radially outwards pressure in a resting condition thereof.

In an exemplary embodiment of the invention, said staves are distortable by an expansion of said balloon.

In an exemplary embodiment of the invention, said balloon is formed of an elastic material.

In an exemplary embodiment of the invention, said plurality of staves are configured to substantially surround said balloon when said balloon is collapsed.

There is also provided in accordance with an exemplary embodiment of the invention, vascular implant, comprising a flexible band having a diameter suitable for implantation in a blood vessel; and a plurality of elongate axial elements mounted on said band. Optionally, said flexible band is thin.

In an exemplary embodiment of the invention, said flexible band has a thickness suitable for restricting blood flow

In an exemplary embodiment of the invention, said flexible band has a length substantially smaller than a length of said elements.

In an exemplary embodiment of the invention, said flexible band is elastic.

There is also provided in accordance with an exemplary embodiment of the invention, a blood flow reducing implant, comprising a body defining a flow channel having an cross-section which is progressively restricted along an axial direction, in which the smallest diameter of a cross-section is sized for passage of a guidewire and blockage of substantially all blood-flow therethrough. Optionally, said cross-section is monotonicly restricted along said direction. Alternatively or additionally, said smallest diameter blocks over 95% of blood flow through said implant. Alternatively or additionally, said smallest diameter is restricted by an elastic sheath.

### BRIEF DESCRIPTION OF THE DRAWINGS

Exemplary non-limiting embodiments of the invention are described in the following description, read with reference to the figures attached hereto. In the figures, identical and similar structures, elements or parts thereof that appear in more than one figure are generally labeled with the same or similar references in the figures in which they appear. Dimensions of components and features shown in the figures are chosen primarily for convenience and clarity of presentation and are not necessarily to scale. The attached figures are:

FIG. 1 is a longitudinal section of a dual wall type flow reducing implant installed in a coronary sinus, in accordance with an exemplary embodiment of the invention;

FIGS. 2A and 2B are isometric views of two embodiments of flap type flow reducing implants, in accordance with an exemplary embodiment of the invention;

FIGS. 3A-3E show various embodiments of cone type flow reducing implants, in accordance with an exemplary embodiment of the invention;

FIG. 4 is longitudinal section of a tube type flow reducing implant, in accordance with an exemplary embodiment of the invention;

FIG. 5 is an isometric view of a staved type flow reducing implant, in accordance with an exemplary embodiment of the invention;

FIGS. 6A-6C are isometric views of three embodiments of wire cone type flow reducing implants, in accordance with an exemplary embodiment of the invention;

FIG. 7 is a longitudinal section of a flow reducing implant with shape-conforming elements, in accordance with an exemplary embodiment of the invention;

FIG. 8A is a plan layout of a ripple type flow reducing implant, in accordance with an exemplary embodiment of the invention;

FIG. 8B is an enlarged section of the plan layout of FIG. 8A, in accordance with an exemplary embodiment of the invention;

FIG. 8C is an isometric view of a slit type flow reducing implant, in accordance with an exemplary embodiment of the invention;

FIG. 9 is a plan layout of a cord type flow reducing implant, in accordance with an exemplary embodiment of the invention;

FIG. 10 is an isometric view of a balloon catheter with expansion rods and a longitudinal section of a flow reducing implant, in accordance with an exemplary embodiment of the invention;

FIG. 11 is an isometric view of a spring ballast catheter and a longitudinal section of a step type coronary sinus flow reducing implant, in accordance with an exemplary embodiment of the invention; and

FIG. 12 shows the step type flow reducing implant of FIG. 11 during manufacture, in accordance with an exemplary embodiment of the invention.

### DETAILED DESCRIPTION OF EXEMPLARY EMBODIMENTS

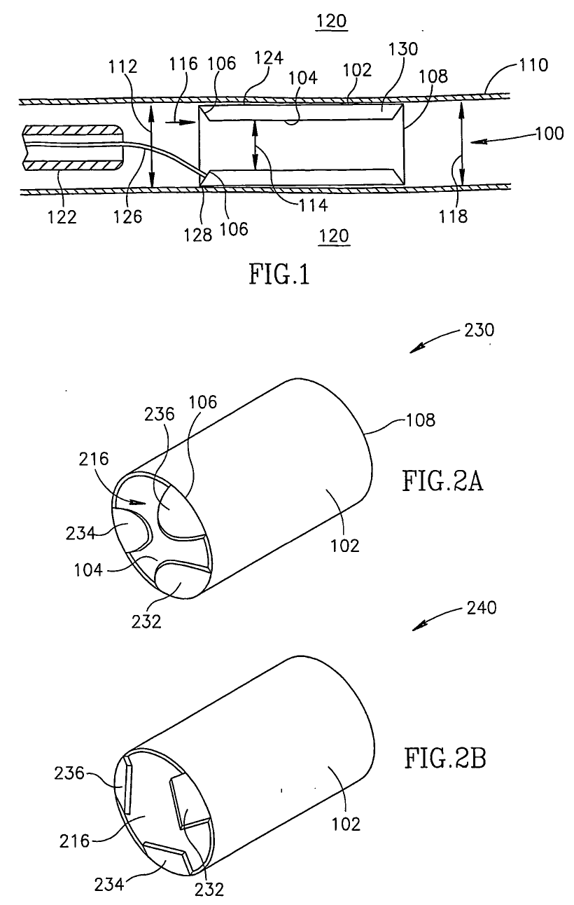

FIG. 1 is a longitudinal section of a dual wall type flow reducing implant 100 installed in a coronary sinus 110 with a pre-implant sinus cross section dimension 112, in accordance with an exemplary embodiment of the invention. Flow reducing implant 100 comprises an outer wall 102 and an inner wall 104. At least a portion of outer wall 102 contacts coronary sinus 110. At least a portion of inner wall 104 is separated from outer wall 102 by a space 130 and defines a flow passage 114 that is narrower in diameter than coronary sinus pre-implant diameter 112.

Thus, blood flowing in a direction 116 will have a reduced flow upon exiting dual wall flow reducing implant 100 via a rear end 108 into a post implant coronary sinus 118. In reducing blood flow in direction 116, flow reducing implant 100 optionally promotes angiogenesis, for example, in an area of coronary tissue 120.

In an exemplary embodiment, inner wall 104 and/or outer wall 102 are resilient and dual wall flow reducing implant 100 is delivered to the deployment site in coronary sinus 110 in a reduced size, for example inside a delivery catheter 122. Upon reaching the deployment area of coronary sinus 110, dual wall flow reducing implant 100 is freed of delivery catheter 122 and expands, for example with pressure from an inflated balloon catheter deployed along flow passage 114.

Alternatively or additionally, a front end 106 and/or rear end 108 are resilient and have a predetermined shape memory so, even with application of an expansion force from a standard balloon catheter, they expand radially outward.

In an exemplary embodiment of the invention, flow reducing implant 100 is cut out of a sheet of metal or a tube, for example, using laser, water cutting, chemical erosion or metal stamping (e.g., with the result being welded to form a tube). Alternatively, flow reducing implant 100 is woven (e.g., of metal or plastic fiber), for example, using methods well known in the art.

In an exemplary embodiment, at least a portion of space 130 is filled, for example with the same material as walls 102 and/or 104, thereby forming a solid wall. Alternatively or additionally, space 130 is filled with, for example, a different material than walls 102 and/or 104. It should be understood that, for example, the self expanding properties (e.g. shape memory) and/or other properties and/or other embodiments of outer wall 102 and/or inner wall 104, apply to any portion of space 130 that serves as a connection between them.

Optionally, shape memory materials of flow reducing implant 100 form directly into the desired shape to provide the required flow reduction, for example, without requiring the use of a catheter balloon. Alternatively or additionally, the catheter balloon used for inflation comprises a single balloon catheter with a standard shape.

Optionally, outer wall 102 is manufactured with a machining process and, for example, etched in a pattern so that a portion of its etched outer surface 124 anchors against coronary sinus 110. Alternatively or additionally, outer surface 124 is fashioned with knobs and/or indentations that promote ingrowth of tissue 120 to provide anchoring of dual wall flow reducing implant 100. Alternatively or additionally, the diameter of outer wall 102 may be varied along its length to conform to contact at least a portion of coronary sinus 110 when coronary sinus 110 has, for example, a variable configuration and/or diameter along its length.

In an exemplary embodiment, flow reducing implant 100 comprises materials that prevent coagulation, embolism formation and/or bacterial colonization. Alternatively or additionally, inner wall 104 and/or outer wall 102 are impregnated with materials that are released over a period of time, for example one month or more or two weeks or less, depending, for example on the patient state of health. These released materials, for example, prevent coagulation, embolism formation and/or bacterial colonization.

Alternatively or additionally, flow reducing implant 100 has a non-cylindrical shape, for example, polygonal or ellipsoid. It may be desirable that flow reducing implant 100 have a non-circular cross-section so that it is less likely to migrate axially. Alternatively or additionally, the cross-section shape and/or orientation optionally change along the length of flow reducing implant 100, for example from a small diameter at end 106 to a large diameter at end 108, or with ends 106 and/or 108 of a small diameter and a large diameter substantially in the center of flow reducing implant 100.

In some embodiments, the surfaces of front end 106 and/or rear end 108 are sloped from coronary sinus 110 toward flow passage 114. Alternatively or additionally, surfaces of front end 106 and/or rear end 108 are sloped toward coronary sinus 110, away from flow passage 114. In still further embodiments, surfaces of front end 106 and/or rear end 108 are perpendicular to outer wall 102. The difference between the various slope designs, for example, may depend on configuration of coronary sinus 110, desired changes in blood flow dynamics and/or preventing an increase in turbulence of blood flow in direction 116 that might result in negative sequella.

In an exemplary embodiment, the diameter of inner wall 104 reduces blood flow 116 of coronary sinus 110 by a specific percentage. In an exemplary procedure used in an embodiment of the present invention, an angiogram of the heart is made that includes the flow through coronary sinus 110. The shape and/or cross sectional diameters of coronary sinus 110 are determined from the angiogram and the size and/or shape and/or configuration of flow reducing implant 100 are determined. In an exemplary embodiment, the outside diameter and configuration of flow reducing implant 100 is closely matched to the inside diameter and configuration of coronary sinus 110 to provide an optimal fit with coronary sinus 110.

In addition to design considerations that allow assumption of an installed shape and/or blood flow reduction, for example, it is desirable to reduce the amount of intrinsic movement that occurs in flow reducing implant 100 and/or other embodiments during expansion. Reducing intrinsic longitudinal movement of flow reducing implant 100 during expansion, for example, reduces potential trauma to the wall of coronary sinus 110 during installation and/or modification in shape of flow reducing implant 100.

Alternatively or additionally, a desired change in the blood volume through coronary sinus 110 is used in determining the configuration of flow reducing implant 100. In an exemplary embodiment, in order to achieve a 50% reduction in blood flow, the cross sectional diameter of coronary sinus 110 is determined from the angiogram and flow reducing implant 100 with an appropriate diameter of narrow passage 114 is chosen to make this reduction.

Alternatively or additionally, inner wall 104 diameter and/or configuration are formed to reduce blood flow to a specific level, regardless of the percentage change of flow reduction. In an exemplary embodiment, outer wall 102 is varied in diameter, for example, to maintain contact with coronary sinus 110. However, narrow passage 114 in many flow reducing implants 100 will have a consistent diameter to restrict blood flow.

Alternatively or additionally, the shape of inner wall 104 and the configuration of front end 106 and/or rear end 108 are altered to change blood flow dynamics and/or promote angiogenesis. Alternatively or additionally, the component materials of flow reducing implant may be altered to promote angiogenesis and/or to reduce untoward reaction in coronary sinus 110.

In an exemplary embodiment of the invention, flow reducing implant 100 is formed of metal, for example, a NiTi alloy (e.g., Nitinol) or stainless steel (e.g., 316L and 316LS). Alternatively, flow reducing implant 100 is formed of, or coated with, other biocompatible materials, such as nylon and/or other plastics. Optionally, flow reducing implant 100 is formed of two or more materials, for example, inner wall 104 being formed of plastic and outer wall 102 being formed of metal.

Coronary sinus 110 is typified by a low degree of elasticity and is relatively susceptible to tearing (as compared to arteries). To provide safe blood flow reduction and/or flow changes that promote angiogenesis, specific considerations must be incorporated into the design of flow reducing implant 100. The design of flow reducing implant 100, therefore, will significantly vary over those associated with, for example, a coronary artery stent.

For example, flow reducing implant 100 may require soft materials and/or soft material coating. Alternatively or additionally, flow reducing implant 100 may require materials with a low spring constant, to prevent flow reducing implant 100 from applying too much pressure on coronary sinus 110. Alternatively or additionally, end 106 and/or end 108 may be coated with a flexible coating, for example a biocompatible material comprising a soft silicone elastomer or another soft plastic or rubber material such as latex, teflon, gortex, kevlar, latex and/or polyurethane to reduce blood flow turbulence, for example.

In an exemplary embodiment, dual wall type flow reducing implant 100 is composed of inflatable material, for example silicone rubber, and upon being freed from delivery catheter 122, it is inflated with an inflator hose 126. Upon completion of inflation, with dual wall type flow reducing implant 100 anchored in coronary sinus 110, for example, inflator hose 126 is pulled free of an inflator seal 128. In an exemplary embodiment, inflator seal 128 automatically seals inflatable dual wall type flow reducing implant 100 to maintain it in the inflated state.

FIG. 2A is an isometric view of a flap type flow reducing implant 230, in accordance with an exemplary embodiment of the invention. Flap type flow reducing implant 230 comprises three flaps 232, 234 and 236 that reduce blood flow in a flow passage 216 and/or promote changes in blood stream dynamics. Three flaps 232, 234 and 236 are shown, though there could be as few as one flap 232, four flaps or more, depending, for example, on the amount of reduction of blood flow and/or change in blood stream dynamics flow that is desired. Flaps 232, 234 and 236 are shown at front end 106 of outer wall 102 though flaps 232, 234 and 236 could be located anywhere along flow passage 216, including rear end 108.

Flaps 232, 234 and 236 are shown projecting forward of front end 106, beyond outer wall 102. Alternatively or additionally, they could all be planar, pointing toward each other and/or perpendicular to outer wall 102. Alternatively or additionally, flaps 232, 234 and 236 could be oblique to outer wall 102 and project into flow passage 216 and/or be located at any position along flow passage 216. Similar positioning of extensions should be understood to apply to other flow reducing implant embodiments.

In an exemplary embodiment, flap type flow reducing implant 230 has inner wall 104 with a reduced diameter compared with the diameter of outer wall 102 in addition to flaps 232, 234 and 236. A reduced diameter increases the reduction in the volume of blood per unit time that passes through flow passage 216 while flaps 232, 234 and 236 change blood stream dynamics.

FIG. 2B is an isometric view of a skewed flap type flow reducing implant 240, in accordance with an exemplary embodiment of the invention comprising three flaps 232, 234 and 236 that are skewed in relation to outer surface 102. A skewed flap type flow reducing implant 240 embodiment may prove to be beneficial in promoting angiogenesis as it changes blood stream dynamics in a robust fashion.

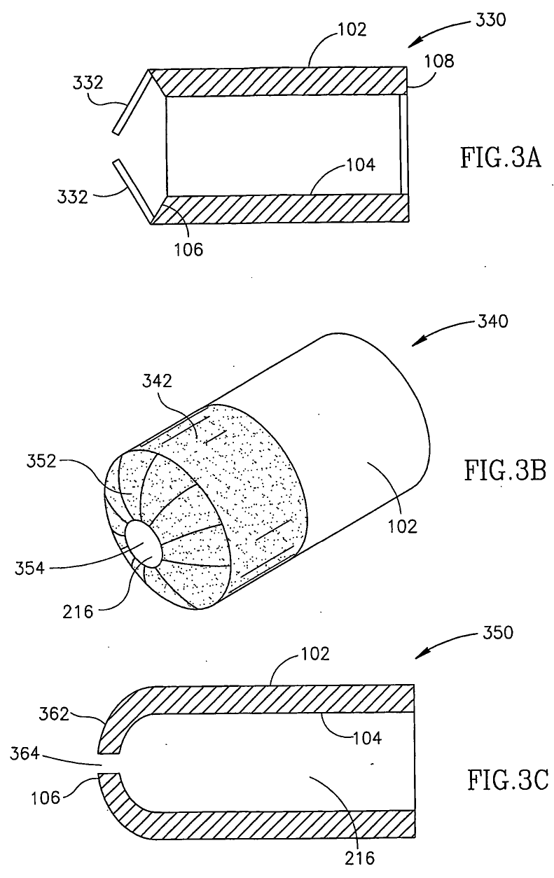

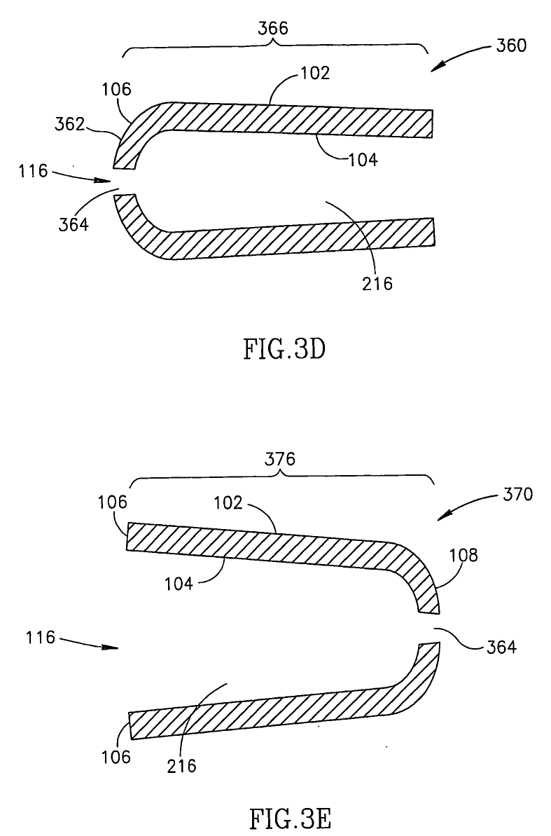

FIGS. 3A-3E show various embodiments of cone type flow reducing implants 330, 340, 350, 360 and 370, in accordance with an exemplary embodiment of the invention. Cone projection type flow reducing implant 340 (FIG. 3A) comprises inner wall 104, outer wall 102 and a cone projection 332 that encircles front end 106. The slope and/or position of cone projection 332 in relation to outer wall 102 and/or inner wall 104 may be varied, in a variety of manners noted above, to alter blood flow dynamics and/or reducing blood flow.

Sheath cone type flow reducing implant 340 (FIG. 3B) comprises a sheath 342 that encircles at least a portion of outer wall 102. Connected to sheath 342 and/or an extension thereof is a sheath projection 352, with an opening 354 to allow passage of blood flow via flow passage 216. Sheath projection 352, for example, can be configured with grooves to control the change in blood stream dynamics in addition to reduction of blood flow.

In an exemplary embodiment, dual cone type flow reducing implant 350 (FIG. 3C) comprises inner wall 104 and outer wall 102 that curve at front end 106 to form a small opening 364, causing reduction in blood flow passage 216. As in dual wall type flow reducing implant 100, dual cone type flow reducing implant 350 can have any combination of expandable and/or inflatable sections to achieve its configuration in the expanded state of FIG. 3C to promote angiogenesis. Dual cone type flow reducing implant 350 comprises thick area between all sections of walls 104 and 106 while dual cone type flow reducing implant 330 comprises cone 332 that is not as thick as the area between walls 102 and/or 104. In an exemplary embodiment, the thick areas between walls 102 and 104 are resilient material for example that is self-expanding. Alternatively or additionally, the thick areas between walls 104 and 104 comprise a space.

As noted, there are a variety of factors that can influence angiogenesis. For example, pressure of sinus blood flow against the valve inlet into the right atrium may favorably influence angiogenesis. Hence the flow pattern of the blood as it leaves coronary sinus 110 to press against the valve leading into the right atrium, may be an important factor in influencing angiogenesis.

To comply with these many scenarios that may serve to promote angiogenesis, dual cone type flow reducing implant 350 may have one or a variety of design variations. For example, the design of front end 106 of dual cone type flow reducing implant 350 and/or its body may be changed to promote flow reduction, change blood stream dynamics and/or increase pressure on coronary sinus 110. Different shapes of outer wall 102 may influence the pressures within coronary sinus 110 to similarly promote angiogenesis.

Additionally or alternatively, for example, front end 106 may be convex in shape around opening 364 to achieve changes blood stream dynamics of blood flowing through coronary sinus 110. Alternatively or additionally, front end 106 may have a flat bevel around opening 364 toward this end. In an exemplary embodiment, dual cone type flow reducing implant 350 is positioned in coronary sinus 110 with opening 364 at its front, facing the blood flow. Alternatively or additionally, dual cone type flow reducing implant 350 is positioned in coronary sinus 110 with opening 364 at its rear, facing away from the blood flow. Optionally, dual cone type flow reducing implant 350 end 106 and end 108 may both be narrowed, for example, to change blood flow dynamics of blood exiting end 108 thereby enhancing angiogenesis.

In an exemplary embodiment, dual cone type flow reducing implant 360 (FIG. 3D) comprises inner wall 104 and outer wall 102 that curve at front end 106 to form small opening 364, causing reduction in the entry of blood flow to passage 216. Alternatively or additionally, inner wall 104 and/or outer wall 102 are tapered to conform for example to coronary sinus 110 as blood flows in direction 116, optionally changing blood flow dynamics to promote angiogenesis. Alternatively or additionally, front end 106 contacts coronary sinus 110 with a strong pressure and a tapered area 366 contacts coronary sinus 110 with a weak pressure and/or does not contact coronary sinus 110 at least along a portion of outer surface 102. In this configuration, for example, the stretch of coronary sinus 110 in the restricted area of front edge 106, may contribute to promoting angiogensis.

In an exemplary embodiment, dual cone type flow reducing implant 370 (FIG. 3E) comprises a sloped area 376 of passage 216 so that front edge 106 comprises the widest diameter of this embodiment of dual cone type flow reducing implant 370. In an exemplary embodiment, blood pressure builds in flow passage 216 and then is released through opening 364, creating a thin stream of blood flow with higher pressure than blood entering flow reducing implant 370 in direction 116. The exiting blood through opening 364 may serve to increase pressure on the valve of coronary sinus 110 that leads into the right atrium. As noted, angiogenesis may be promoted by blood flow changes that affect the valve of the atrium. Alternatively or additionally, a taper along area 376 may be appropriate to conform, for example, to coronary sinus 110 that itself tapers from a wider cross sectional diameter to a narrower cross sectional diameter.

In an exemplary embodiment of the invention, opening 364 is made very small, for example substantially blocking all blood flow therethrough, such as over 90%, 95% or 98% of such flow being blocked by the reducer. However, an opening 364 may still be useful, for example for mounting on a guide wire. For example, opening 364 may be sized to receive (with a small amount of freedom), a guidewire having a diameter, of, for example, 14/1000 of an inch or smaller, such as 7/1000 of an inch, or larger, such as 20/1000, 30/1000 or 40/1000 of an inch. Alternatively or additionally, a sheath 352 as in FIG. 3B is used, except that its aperture 354 is normally closed, but is elastic and allows the passage of a guidewire therethrough. Such an elastic sheath may also be provided on aperture 364.

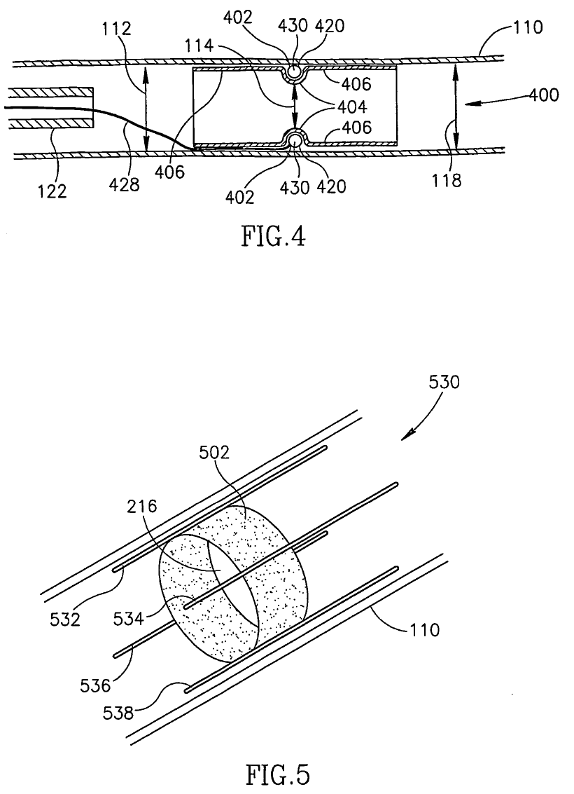

To achieve blood flow that promotes angiogensis, a relatively rapid transition from (wide) pre-implant diameter 112 to narrow passage 114 and return to (wide) post implant diameter 118, (FIG. 4) may prove to promote angiogenesis, for example, due the change in flow dynamics it creates. A tube type flow reducing implant 400, in accordance with an exemplary embodiment of the invention, comprises a long wall 406, a portion of which is surrounded by a ring-shaped tube 420. Optionally, the diameter of flow passage 114 adjacent a bulge 404 in long wall 406 provides a rapid transition from pre-implant diameter 112 to narrow passage 114 and back to sinus post implant diameter 118.

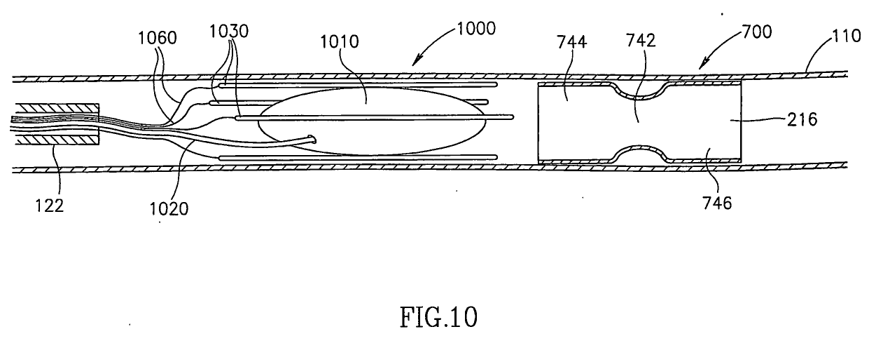

In an exemplary embodiment, tube 420 has an interior space 430 enclosed within a circular wall 402 that is, for example, inflatable using a hose 428, for example, in a similar fashion to hose 1020 in FIG. 10, explained below.

In an exemplary embodiment, tube 420 inflates so that interior 430 has two or more cross sectional diameters, thereby allowing adjustment of narrow passage 114 to modify the amount of reduction in blood flow and/or other factors of blood flow, for example, change blood stream dynamics.

Alternatively or additionally, interior 430 contains a material that absorbs liquid, thereby expanding. Following implantation, for example, tube 420 absorbs liquid and interior 430 increases in size until tube 420 reaches its expanded state.

Alternatively or additionally, wall 402 and/or tube 430 comprise a resilient material, for example Nitinol, and expand to a final state without inflation. Alternatively or additionally, flow reducing implant 400, and/or embodiments mentioned below, are manufactured from a biocompatible material, comprising, for example, a soft silicone elastomer and/or another soft material such as latex, teflon, gortex, kevlar and/or polyurethane.

Alternatively or additionally, interior 430 is filled, for example with a spongy material, for example that is different than the material comprising long wall 406 and/or wall 402. Spongy material of interior 430, for example remains compressed in a compact size until its exit from catheter 122 whereupon interior 430 expands, causing the expansion of tube 420.

In an exemplary embodiment, long wall 406 is contoured and comprises a shape memory material and achieves its final state, including bulge 404, upon exit from catheter 122. Alternatively or additionally, long wall 406 is, for example, not contoured and tube 420 presses against long wall 406 to create bulge 404.

FIG. 5 is an isometric view of a staved type flow reducing implant 530, in accordance with an exemplary embodiment of the invention, comprising staves 532, 534, 536 and 538 around a resilient membrane wall 502. Resilient membrane wall 502 of staved type flow reducing implant 530 is of a material and a thickness that allow it to readily project into flow passage 216 upon the movement of staves 532, 534, 536 and 538 toward each other. As flow reducing implant 530 assumes a compact state without, for example, trailing resilient material 502, staved type flow reducing implant 530 is easily positioned inside catheter 122 (FIG. 1) for removal and/or repositioning in coronary sinus 110.

Alternatively or additionally, staved type flow reducing implant 530 is at least partially reduced in diameter by the pressure of the inner surface of coronary sinus 110 as it is moved longitudinally to a new position in coronary sinus 110, with membrane wall 502, for example, projecting into flow passage 216. In an exemplary embodiment, of staves 532, 534, 536 and 538 and/or resilient material 502 comprise shape memory materials and, after attaining its new position in coronary sinus 110, flow reducing implant 500 returns to its memorized shape.

Alternatively or additionally, a balloon catheter is deployed, for example, to cause staved type flow reducing implant 530 to assume is memorized shape after it has reached its new position in coronary sinus 110.

In a particular embodiment of the invention, membrane 502 is formed of or coated with a material that enhances adhesion thereto, for example PTFE or a tissue adhesive, at least on its outer surface. In this embodiment, a thin membrane may be used, with the narrowing effect achieved by the collapsing of the vessel on the membrane instead of or in addition to any effect of the thickness of the membrane. Optionally, the staves are pre-stressed so that one or both of their outer ends project radially outwards. Optionally, this pre-stressing assists in anchoring in—and/or collapsing of—the blood vessel. Optionally, the ends of the staves are made rounded, for example in the form of rounded plates, to prevent inadvertent penetration. Alternatively or additionally, the staves are replaced by a stent and/or have stent sections at one or both ends.

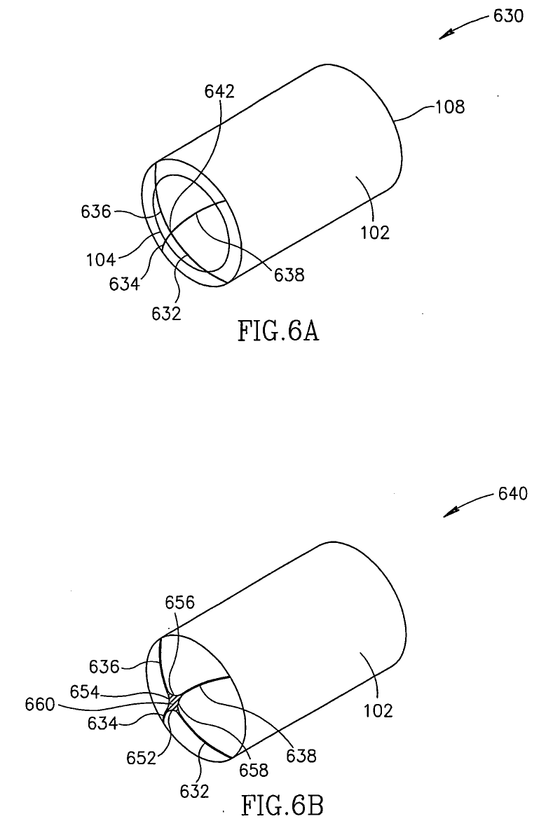

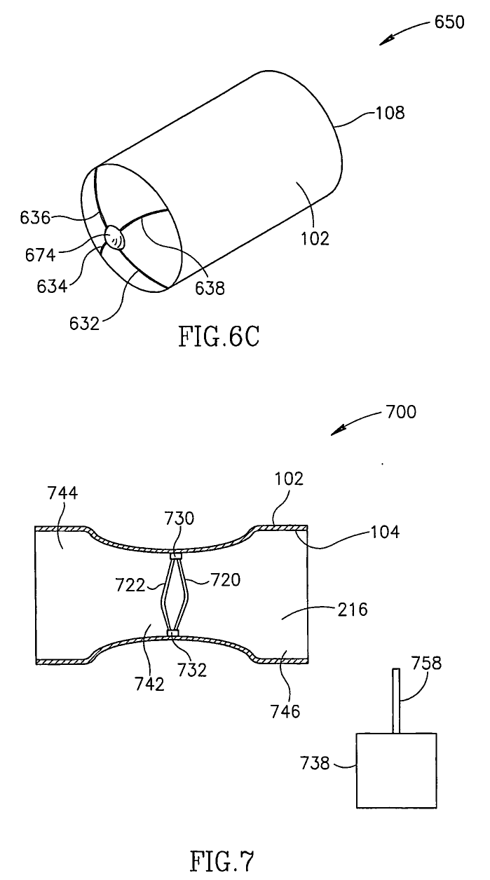

FIG. 6A is an isometric view of a wire cone type flow reducing implant 630, in accordance with an exemplary embodiment of the invention, comprising one or more transverse wires 632, 634, 636 and/or 638 spanning flow passage 216 to reduce blood flow and/or change blood stream dynamics. Wires 632, 634, 636 and/or 638 may be, for example, joined at a point 642 and may be curved and/or straight in one or more projection planes in relation to wire cone type flow reducing implant 630 to reduce blood flow and/or change blood stream dynamics

In an exemplary embodiment, elements 632, 634, 636 and/or 638, for example, form two continuous wires comprising wire 632 continuous with wire 636 and/or wire 634 continuous with wire 638. Alternatively, wires 632, 634, 636 and/or 638 may be separate from each other, but bent so that their tips come close to one another near point 642. Optionally, wire 632 continuous with wire 636 may be straight. Alternatively or additionally, wire 632 continuous with wire 636 may be bowed, for example, so the bow extends beyond outer wall 102 and/or inside inner wall 104, thereby influencing blood stream dynamics to initiate and/or increase angiogenesis.

Alternatively or additionally, as with flaps 232, 234 and 236, wires 632, 634, 636 and/or 638 are shaped to extend beyond, perpendicular to and/or interior to flow passage 216. Similarly, wire cone type flow reducing implant 630 may have, for example, an outer wall 102, inner wall 104 and/or space 430 configured in similar fashion to other embodiments described.

In laminar blood flow dynamics, the blood that is closest to the inner walls of coronary sinus 110 move more slowly than blood flow passing through the center of coronary sinus 110. It may be desirable to further slow the blood flow in the center of coronary sinus 110 over that provided by wires 632, 634, 636 and/or 638, thereby promoting angiogenesis.

FIG. 6B is an isometric view of a plate wire cone type flow reducing implant 640, in accordance with an exemplary embodiment of the invention, comprising one or more transverse wires 632, 634, 636 and/or 638 that are joined to a plate 660 spanning flow passage 216. Plate 660, for example, is positioned to block blood flow in the center of coronary sinus 110. Further changes in the configuration of transverse wires 632, 634, 636 and/or 638, and/or plate 660, for example so they are thicker and/or of variable thickness, are contemplated for the purpose of modifying the blood flow pattern to promote angiogenesis.

In an exemplary embodiment, plate 660 comprises four curves, 652, 654, 656 and/or 658 to which wires 632, 634, 636 and/or 638 are joined thereby providing a connection between plate 660 and wires 632, 634, 636 and/or 638 that reduces turbulence in blood flow. Alternatively or additionally, plate 660 may comprise a passage through its center, for example being round in shape, thereby further modifying the flow pattern of blood through coronary sinus 110.

FIG. 6C is an isometric view of a sphere wire cone type flow reducing implant 650, in accordance with an exemplary embodiment of the invention, comprising one or more transverse wires 632, 634, 636 and/or 638 that are joined to a spherical member 674 spanning flow passage 216. Spherical member 674, for example, may comprise a variety of sizes and/or shapes such as flat spheroid, ovoid and/or others, depending, for example, on amount of flow reduction required, angiogenesis promotion and/or flow turbulence reduction.

In an exemplary embodiment, wires 632, 634, 636 and/or 638 of wire cone type flow reducing implants 630, 640 and/or 650, are resilient so that they automatically bow into their final position shown in their respective figures upon exiting catheter 122 (FIG. 1). Alternatively or additionally, wires 632, 634, 636 and/or 638 of wire cone type flow reducing implants 630, 640 and/or 650, may comprise flexible materials and/or flexible chains that assume their final shape dependent upon, for example, their drag in the blood flowing around them.

In an exemplary embodiment, wire cone type flow reducing implants 630, 640 and/or 650 may be reduced in size with wires 632, 634, 636 and/or 638 and/or their attachments, for example spherical member 674, beyond outside wall 102. The less material contained between inside walls 104, for example, allows outer wall 102 to assume a smaller diameter when in a reduced size, thereby facilitating removal and/or repositioning in a portion of the coronary sinus that has a smaller diameter. This is particularly useful when coronary sinus 110 is of a narrow diameter. Alternatively or additionally, spherical member 674 and/or wires 632, 634, 636 and/or 638 position inside of outside wall 102, following reduction in size, to prevent possible trauma as they are moved-against the walls of coronary sinus 110.

To promote angiogenesis, as noted, it may be necessary to change the configuration of a flow reducing implant 700 and/or another of the other embodiments of flow reducing implant 100 following implantation in coronary sinus 110. Changes in the configuration of flow reducing implant 700, for example, may change the blood flow pattern and/or flow volume in coronary sinus 110 to further promote angiogenesis and/or prevent untoward sequella due to improper blood flow turbulence. The necessary changes in the configuration of flow reducing implant 700, for example, may require delicate manipulation of the various flow reducing implant embodiments.

FIG. 10 is an isometric view of a balloon catheter 1000 with expansion rods 1030 in accordance with an exemplary embodiment of the invention, that facilitates fine adjustments in the configuration of a flow reducing implant 700 shown in a longitudinal section. In an exemplary embodiment, balloon catheter 1000 comprises a balloon 1010 connected to a hose 1020 that inflates and/or deflates balloon 1010.

In an exemplary embodiment, balloon catheter 1000 is used to open and/or modify the shape of type flow reducing implant 700. For example, balloon catheter 1000 is positioned within a front flare 744 and inflated using inflator hose 1020 thereby expanding its rods 1030 radially outward to exert pressure on front flare 744 to cause its expansion. Following this, balloon catheter 1000 is deflated using inflator hose 1020.

In an exemplary embodiment, rods 1030, for example, are positioned around balloon 1010 that comprises a material of a flexibility and a thickness that allow it to readily reduce in diameter between rods 1030 upon deflation. With balloon 1010 contained between rods 1030, balloon catheter 1000 easily passes through a narrow passage 742, into a rear flare 746.

With balloon catheter 1000 positioned within rear flare 746, it is inflated using inflator hose 1020 thereby expanding its rods 1030 radially outward to cause expansion of rear flare 746. Following this, balloon catheter 1000 is deflated through inflator hose 1020 to pass into narrow passage 742 where it is inflated to cause expansion of passage 742. Balloon catheter 1000 is then deflated and moved to front flare 744 and inflated to cause expansion of flare 722. Finally balloon catheter 1000 is deflated and removed from coronary sinus using, for example, a percutaneous catheter removal technique known in the art.

In an exemplary embodiment of the invention, once flow reducing implant 700 is formed, it is mounted on a jig having the desired final expanded shape and heated so that it naturally attains that shape, for example, when released from catheter 122. In an exemplary embodiment, narrow passage 742 is manufactured using a different material and/or process than that of flares 744 and/or 746. For example, flares 744 and/or 736 are woven into a mesh and narrow passage 742 is cut from sheet metal.

In an exemplary embodiment, flare ends 744 and/or 746 have a diameter of between 2 mm and 30 mm, for example, 5 mm, 10 mm, 15 mm, 20 mm or any larger, smaller or intermediate diameter, for example selected to match the diameter of coronary sinus 110. Narrow passage 742 diameter may be, for example, 1 mm, 2 mm, 3 mm, 5 mm, 10 mm or any smaller, larger or intermediate diameter, for example selected to achieve desired flow dynamics and/or a pressure differential across flow reducing implant 700.

In an exemplary embodiment of the invention, the ratio between the cross-section of narrow passage 742 and flare end 744 and/or flare end 746 is 0.9, 0.8, 0.6, 0.4, 0.2 or any larger, smaller or intermediate ratio, for example selected to achieve desired flow dynamics and/or a pressure differential across flow reducing implant 700.

Changing the configuration, for example, of flow reducing implant 700 using, for example, balloon catheter 1000 may be desired, for example, to alter the blood flow volume and/or blood stream dynamics. However, such change involves invasion of the patient's circulatory system and care must be taken not to disrupt the heart's blood supply and/or rhythm, particularly in patients with compromise coronary circulation. In an exemplary embodiment, modification of flow reducing implant 700, in order to expand and/or reduce the size of narrow area 742 and/or flares 744 and/or 746 may be accomplished without invasion of the vasculature.

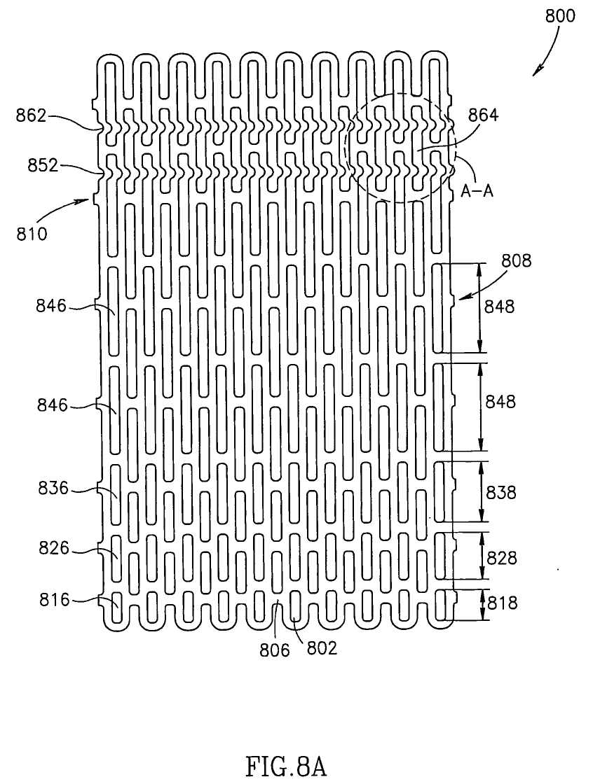

FIG. 7 is longitudinal section of flow reducing implant 700, in accordance with an exemplary embodiment of the invention, comprising one or more shape-conforming elements 720 and/or 722 that can be remotely induced to change their configuration. Remote control of the configuration of elements 720 and/or 722 causes, for example, change in configuration, constriction and/or expansion of narrow passage 742, and/or flares 744 and 746 without associated hazards of an invasive procedure. As narrow passage 742 and/or flare 744 and/or flare 746 change their configuration, the blood flow dynamics are altered, thereby promoting angiogenesis. Alternatively or additionally, as narrow passage 742 and/or flare 744 and/or flare 746 constrict and/or expand, the blood flow pattern in coronary sinus 110 changes, thereby influencing angiogenesis.

Shape-conforming elements 720 and/or 722, for example, are charged so that as they receive impulses from impulses 730 and/or 732, they change into one or more different geometric shapes and/or configurations. The shapes of elements 720 and/or 722 induced by impulsers 730 and 732 cause changes in the configuration of blood flow reducing implant 700, thereby influencing angiogensis.

For example, one or both shape-conforming elements 720 and/or 722 straighten, they exert outward expansion pressure on narrow passage 742, thereby allowing blood flow therethrough to increase. When one or both shape-conforming elements 720 and/or 722 bend further than depicted in FIG. 7, they pull the walls of narrow passage 742 inward, causing passage 742 to narrow, thereby reducing blood flow therethrough.

Alternatively or additionally, when shape-conforming elements 720 and/or 722 bend or straighten the walls of narrow passage 742 may change its configuration, thereby causing changes in blood stream dynamics and/or pressure of blood flow along flow passage 216 and into coronary sinus 110, all of which may influence angiogenesis.

Alternatively or additionally, shape-conforming elements 720 and/or 722 are located exterior to flow reducing implant 700, for example along outer wall 102. Alternatively or additionally, other shape-conforming elements 720 and/or 722 may be located along flares 744 and/or 746 to provide additional and/or alternative remote control of flow reducing implant 700.

Optionally, impulses provided by impulsers 730 and 732 to induce changes in shape-conforming elements 720 and/or 722 and comprise one or more of: RF, acoustic waves such as ultrasound and/or low frequency sound, heat, electricity, electromagnetic, radiation. Alternatively or additionally, impulsers 730 and 732 mediate a chemical reaction that modifies elements 720 and/or 722, thereby changing their configuration.

In an exemplary embodiment, a director 738, external to the patient, directs impulsers 730 and 732 to provide impulses to shape-conforming elements 720 and/or 722, thereby causing the changes in geometric shape. Director 738, for example, directs impulsers 730 and 732 via radio waves from an antenna 758.

Alternatively or additionally, elements 720 and/or 722 are sensitive to waves that are propagated external to the patient For example, director 738 provides one or more of: RF, acoustic waves such as ultrasound and/or low frequency sound, heat, electricity, electromagnetic and radiation to influence the configuration of elements 720 and/or 722.

In an exemplary embodiment, shape-conforming elements 720 and/or 722 comprise a material with a positive charge, for example positively charged plastic and/or silicone rubber. Alternatively or additionally, shape-conforming elements 720 and/or 722 comprise a negatively charged material.

Optionally, shape-conforming elements 720 and/or 722 are manufactured from a material comprising charged lithium ions. In an exemplary embodiment, waves cause the charged lithium ions to align, thereby changing the geometry of shape-conforming elements 720 and/or 722 to cause changes in the shape of outer wall 102 and/or inner wall 104.

In an exemplary embodiment, the strength and/or length of impulses aid in changing shape-conforming elements 720 and/or 722. For example, impulsers 730 and 732 provide an electric impulse of between 0.1 volts and 0.5 volts (optionally, 0.1 volts or less or 0.5 volts or more), for a period of 10 msec or longer or 6 msec. or shorter. The factors influencing the impulse chosen, for example, depend upon materials comprising shape-conforming elements 720 and/or 722, their responsiveness to the impulses and/or the desired changes in their shapes to influence the shape of flow reducing implant 700.

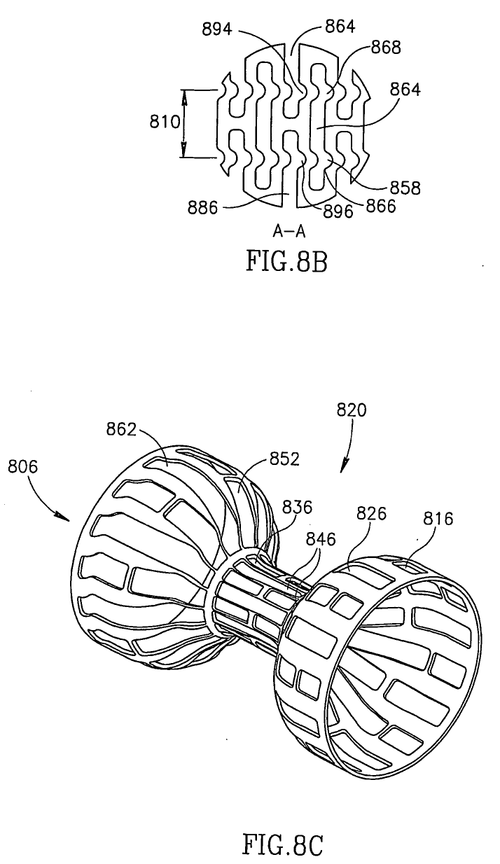

Flow reducing implant 700, with shape-conforming elements 720 and/or 722 allows modification in shape and/or blood flow reduction following implantation of flow reducing implant 700 in coronary sinus 110 without an invasive procedure. Alternatively or additionally, an embodiment of flow reducing implant 700 that assumes its installed shape without for example, the use of balloon catheter 1000 may be desirable. In an exemplary embodiment of the present invention, ripple type flow reducing implant 800 (FIGS. 8A-8C) comprises shape memory materials that automatically achieve a final configuration state upon exiting catheter 122, thereby averting the use of balloon catheter 1000 for initial installation of ripple type flow reducing implant 800.

Alternatively or additionally, ripple type flow reducing implant 800 contains preformed rows of ripples 852 and/or 862. Ripples 852 and/or 862 allow modification in size and/or configuration of ripple type flow reducing implant 800 with a minimal amount of expansion force and/or a minimal amount of time using balloon catheter 1000. Reduction in time and/or force with balloon catheter 1000, reduces the risk of untoward sequella, for example, to the patient with compromised vasculature.

FIG. 8C is an isometric view of a slit type flow reducing implant 820, in accordance with an exemplary embodiment of the invention, comprising rows of slits 816, 826, 836 and 846.

FIG. 8A is a plan layout of ripple type flow reducing implant 800 whose details are somewhat different from that of the slit type reducing implant 820 shown in FIG. 8C. Ripple type flow reducing implant 800 has a row of ripples 862 and a row of ripples 852, corresponding to slit rows 862 and 852 respectively in slit type flow reducing implant 820. Further, for the representation, ripple type flow reducing implant 800 has been cut to separate an edge 810 from an edge 808, thereby providing its plan view.

Ripple type flow reducing implant 800 comprises longitudinal rows of slits 816, 826, 836 and 846, having lengths of 818, 828, 838 and 848 respectively. In an exemplary embodiment, rows of slits 816, 826, 836 and 846, for example, automatically expand to form installed ripple type flow reducing implant 800 without use of balloon catheter 1000. Ripple type flow reducing implant 800 comprises an outer surface (not shown) and an inner surface 802 that defines a flow passage 806 that is shaped, for example, in a similar shape as that of flow reducing implant 700.

Ripple type flow reducing implant 800, for example, attains a final shape that is, for example, similar to that of flow reducing implant 700. This final shape, for example, occurs as its shape memory material expands when released from catheter 122 (FIG. 1). Alternatively or additionally, balloon catheter 1000 may be used to facilitate expansion of ripple type flow reducing implant 800, for example, when it is made of materials without an automatic shape memory. However, rows of slits 816, 826, 836 and 846 with their lengths and/or orientation that promote a specific final shape, allow ripple type flow reducing implant 800 to readily form into a final configuration even when not formed of shape memory materials. Therefore, installation of ripple type flow reducing implant 800 optionally occurs with a minimal amount of time and/or expansion force by balloon catheter 1000.

In an exemplary embodiment, flow passage 806 corresponds to flow passage 216 in FIG. 10, comprising at least two diameters, a small diameter corresponding to slits 846 and a flared diameter corresponding to slits 836, 826 and/or 816.

In an exemplary embodiment, ripple type flow reducing implant 800 may easily be further modified as it contains two rows of ripples 852 and 862 that, for example, expand flow passage 806 in response to expansion pressure from balloon catheter 1000. When balloon catheter 1000 is introduced into flow passage 806 and expanded, rows of ripples 852 and/or 862 are induced to straighten, thereby increasing the diameter of flow passage 806 through ripple type flow reducing implant 800.

Alternatively or additionally, the apex of each ripple in ripple row 852 face into flow passage 806, and the apex of each ripple of ripple row 862 face away from flow passage 806. In an exemplary embodiment, ripple row 852 expands at a first expansion pressure from balloon catheter 1000 as the apex of each ripple of ripple row 852 contact expansion balloon catheter 1000 as it expands against surface 802.

In an exemplary embodiment, ripple row 862 expands with application of a second expansion pressure as the apex or each ripple in ripple row 862 does not come in contact with balloon catheter 1000 and hence only pressure of balloon catheter 1000 on flow passage 806 causes their expansion.

In this embodiment, for example, an initial pressure of between 34 atmospheres (optionally 3 atmospheres or less or 4 atmospheres or more) causes expansion of ripple row 852. A second pressure, for example, of between 7-8 atmospheres (optionally 7 atmospheres or less or 8 atmospheres or more), causes the expansion of ripple row 862.

Alternatively or additionally, the apex of each ripple of ripple row 862 face the same way as the apex of each ripple of ripple row 852 and ripple row 862 comprises a material, material coating and/or material additive that renders it stiffer, for example, than ripple row 852. As a result of the change in material of ripple row 862, for example, ripple row 862 does not expand when a lower expansion pressure, sufficient to expand ripple row 852 is applied to flow passage 806.

Ripple type flow reducing implant 800, demonstrates easy implantation without using, for example, balloon catheter 1000 for implantation due to its shape memory. In addition, modification of ripple type flow reducing implant 800 following implantation is easily and/or rapidly accomplished using balloon catheter 1000 that presses against one or more ripple rows 852 and/or 862.

FIG. 8B is an enlarged of the plan layout of ripple type flow reducing implant 800, in accordance with an exemplary embodiment of the invention. Section A-A comprises a slot 858 with a first radius 864 of 0.2 millimeters and a second radius 866 of 0.2 millimeters though radii 864 and/or 866 could be between 0.1-0.3 millimeters (optionally 0.1 millimeters or smaller or 0.3 millimeters or larger) depending upon, for example, the materials used and/or their flexibility. A distance between radii 864 and 866, for example, is 1.0 millimeters though it could be between 0.5-2.0 millimeters (optionally 0.5 millimeters or smaller or 2.0 millimeters or larger), depending, for example on the contour of ripple type flow reducing implant 800.

Additionally, section A-A comprises a left slot 884 and a right slot 886. In an exemplary embodiment, left slot 884 has a ripple with a left radius 894 of 0.2 millimeters and right slot 886 has a ripple 896 with a right radius 896 of 0.2 millimeters. Additionally or alternatively, radii 894 and/or 896 could be between 0.1-0.3 millimeters (optionally 0.1 millimeters or smaller or 0.3 millimeters or larger) depending, for example on the materials used and/or their flexibility.

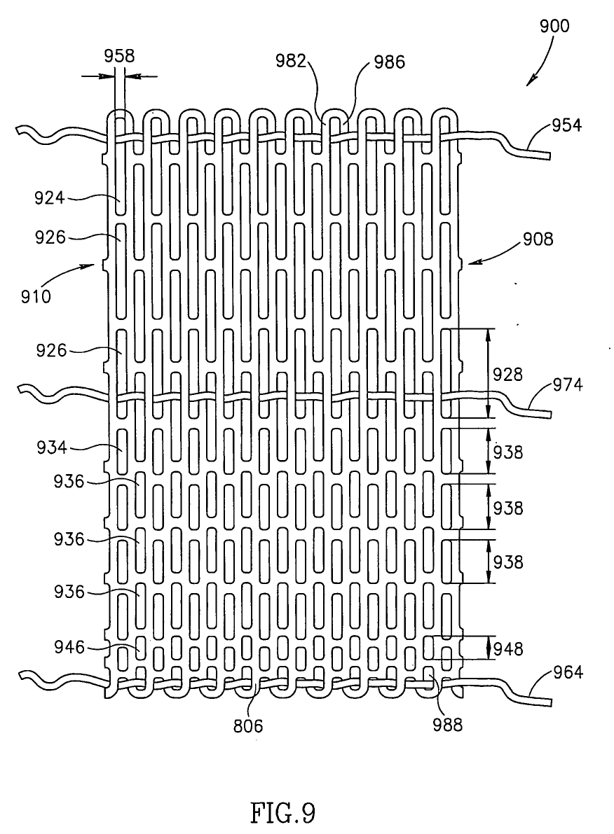

In an exemplary embodiment of the present invention, a cord type flow reducing implant 900 shown in a plan view in FIG. 9, comprises a preformed shape that, like ripple row type flow reducing implant 800, allows it to easily spring into its installed shape without, for example, use of balloon catheter 1000. In an exemplary embodiment, one or more edges 910 are joined to one or more edges 908 to form cord type flow reducing implant into a tubular shape with flow passage 806 passing therethrough.

In its assembled state, cord type flow reducing implant 900 comprises a row of slits 924 through which a cord 954 passes, that is modified with minimal expansion pressure from balloon catheter 1000.

In an exemplary embodiment, cord 954 is woven to pass under a lead post 982 and over a trailing post 986 so that cord 954 is woven across cord type flow reducing implant 900. Alternatively or additionally, cord 954 is expandable and attached to surfaces of slots 924, for example their surfaces facing flow passage 806 or their opposite (outside) surfaces.

Alternatively or additionally, cord 954 of cord type flow reducing implant 900 is expandable to allow modification in the shape of cord type flow reducing implant 900, on one or more additional occasions. Repeated modification of cord type flow reducing implant 900 may be desirable, for example, for the patient with unstable coronary vascular flow.

In an exemplary embodiment, cord type flow reducing implant 900 automatically assumes is memorized shape upon exiting catheter 122 as slits 926, 936 and/or 946 automatically expand. In an exemplary embodiment, cord 954 passes through row of slits 924 and has a thickness that creates a bulge in flow passage 806, thereby creating a narrowing in flow passage 806 that changes blood flow dynamics, for example.

In an exemplary embodiment, after cord type flow reducing implant 900 expands to its initial configuration automatically upon exiting catheter 122 and further size modification is required, balloon catheter 1000 is introduced into the interior of cord type flow reducing implant 900. Balloon catheter 1000 is inflated, for example, between 3-4 atmospheres (optionally, 3 atmospheres or less or 4 atmospheres or more), and causes row 924 to move radially outward against cord 954. Cord 954 moves radially outward, thereby smoothing the bump that cord 954 causes in flow passage 806 along row of slits 924, changing the flow dynamics of the blood flow through flow passage 806.

In an exemplary embodiment, at least a portion of an edge 910 is detached from at least a portion of an edge 908 so when flow reducing implant 900 forms its expanded shape, for example, at least a portion edge 910 and edge 908 overlap. If further expansion is required, additional expansion force is applied, for example, between 7-8 atmospheres (optionally, 7 atmospheres or less or 8 atmospheres or more) of pressure and cord 954 elongates so that edge 910 draws closer and/or passes edge 908, allowing cord type flow reducing implant 900 to attain another, expanded, diameter.

In an exemplary embodiment, cord 954 comprises a plastic material that stretches to two or more lengths, depending upon the expansion pressure that is applied to it. Hence, at a lower pressure, cord 954 expands to a first length, thereby defining a first narrow diameter of cord type flow reducing implant 900. Subsequently a second expansion pressure is applied and cord 954 attains a second, longer, length, thereby defining a second diameter, wider than the narrow diameter.

Alternatively or additionally, cord type flow reducing implant 900 includes one or more diameters in which edge 910 and edge 908 are separated by a space, thereby providing an interrupted flow passage surface 802. Alternatively or additionally, cord 954 severs upon application of, for example, pressure between 9-10 atmospheres (optionally 9 atmospheres or less or 10 atmospheres or more). Upon severing cord 954, edge 910, for example, maximally separates from edge 908, thereby applying unrestricted pressure against coronary sinus 10. As noted above, increased pressure on coronary sinus 110 may enhance angiogenesis caused by one or more other factors.

In an exemplary embodiment, cord 954 of flow reducing implant 900 comprises a biocompatible material that dissolves in the environment of coronary sinus 110, for example, a material comprising galactic acid and/or polygalactic acid and/or other materials with similar properties. In an exemplary embodiment, flow reducing implant 900 is placed in coronary sinus 110 and balloon catheter 1000 is used to expand it so that its outer surface contacts the inside surface of coronary sinus 110. Over a period of time, for example three days or less or four days or more, cord 954 degrades, depending upon the biodissolvable material comprising cord 954. Once cord 954 has dissolved, flow reducing implant 900 retains its shape, with its outer surface in contact with the inner surface of coronary sinus 110.

With cord 954 dissolved, further expansion of inner diameter of flow reducing implant 900 is accomplished with balloon 1010 at a low atmospheric pressure due to the fact that edge 908 passes edge 910 without the hindrance of cord 954. Hence, to cause edge 908 to pass edge 910, expansion force need only overcome the stiffness of the material comprising flow reducing implant 900. In an exemplary embodiment, a pressure of between 34 atmospheres (optionally 3 atmospheres or less or 4 atmospheres or more), causes expansion of wall the flow passage through flow reducing implant 900.

In an exemplary embodiment of the present invention, flow reducing implant 900 comprises cord 954 passing through slits 924 and a cord 964 passing through slots 988. Alternatively or additionally, flow reducing implant 900 comprises three or more cords 954, 964 at either end and a cord 974 passing through slots 926 substantially in the middle of flow reducing implant 900.

Cords 954, 964 and/or 974 serve to maintain the shape and/or appropriate flow passage diameter following installation. To expand the flow passage through flow reducing implant 900, balloon catheter 1000 is used to expand and/or sever cords 954, 964 and/or 974. Alternatively or additionally, sever cords 954, 964 and/or 974 are biodissolvable, dissolving in the environment of coronary sinus 110.

In an exemplary embodiment, when cord type flow reducing implant 900 is configured according to the shape of flow reducing implant 700 (FIG. 10), little or no blood migrates through narrow passage 742, flare 744 and/or flare 746 to contact the walls of coronary sinus 110. This, for example, is achieved by the narrow cross-section and/or configuration of slits 936 and/or 946 to limit and/or prevent migration of blood through the walls of narrow passage 742, flare 744 and/or flare 746. In an exemplary embodiment, to achieve this limitation of blood migration with adequate expansion of cord type flow reducing implant 900, slits 938 and/or 948 are increased in number, while the width of slits 926, 936 and/or 946 is reduced.

In a particular example, only the widths of slits 926 are reduced, thereby increasing the amount of material near the center of the implant and making the center more difficult to expand, relative to the flared ends.

Alternatively or additionally, an elastic coating is provided on the inside and/or outside of flow reducing implant 700, for example, latex, to prevent flow through openings slits 938 and/or 948. In an exemplary embodiment of the invention, the coating is a separate, flexible layer, that is attached to flow reducing implant 700 at one or more points (e.g., at narrow passage 742 and/or flare 744 and/or flare 746) to prevent tearing of the layer by the expanding flow reducing implant 700. Alternatively or additionally, the coating is preformed to the shape of the expanded flow reducing implant 700. Prior to expansion, for example, this coating layer is folded and/or pleated.

In an exemplary embodiment of the present invention, the material thickness for the walls of flow reducing implant 900 and/or other flow reducing implant embodiments, is 0.15 mm. However thinner or thicker materials may be used dependent upon factors such as strength of materials and/or flow dynamic changes desired.

In an exemplary embodiment, flow reducing implant 900 is designed to shorten minimally during installation, for example, having a length of 20 mm before installation and about 18.8 mm after installation. Alternatively or additionally, a non-shortening design is used, for example a mesh as in peristaltic stents, such as described in U.S. Pat. No. 5,662,713, the disclosure of which is incorporated herein by reference.

The length of installed flow reducing implant 900 and other embodiments, for example, is optionally selected to match a physiological size of the target vein (e.g., length and curves) and/or to ensure good contact with vein walls. Exemplary lengths are 5 mm, 12 mm, 24 mm, 35 mm 45 mm and any smaller, intermediate or larger size. Alternatively or additionally, the length of narrow passage 742 (FIG. 7), for example, may be 0.5 mm, 1 mm, 2 mm, 3 mm, 5 mm or any smaller, intermediate or larger length, for example selected to achieve desired flow dynamics.

FIG. 11 is an isometric view of a spring ballast 1100 and a longitudinal section of a step type flow reducing implant 1180 in accordance with an exemplary embodiment of the invention. Spring ballast 1100 comprises spring rods 1130 that are, for example curved and attached to each other at least one end 1192. In an exemplary embodiment, spring rods 1130 expand radially outward upon exiting catheter 122. In an exemplary embodiment, catheter 122 is placed at narrow passage 114 of step type flow reducing implant 1180. Spring ballast 1100 exits catheter 122, passes through narrow passage 114 past a rear passage 1156 into coronary sinus 110 with post implant diameter 118.

In an exemplary embodiment, spring ballast 1100 is pulled with tethers 1060 in a direction 1062 toward rear passage 1156. Spring ballast 1100 reduces in size between a wall 1146 surrounding passage 1156 and the radial outward pressure caused by spring rods 1130 on wall 1146, causes expansion of wall 1146 into its expanded position.

In an exemplary embodiment, spring ballast 1100 is pulled with tethers 1060 in direction 1062 toward narrow passage 114. Spring ballast 1100 reduces in size between a wall 1142 surrounding narrow passage 114 and the radial outward pressure caused by spring rods 1130 on wall 1142, causes expansion of wall 1142 into its expanded position.

In an exemplary embodiment, spring ballast 1100 is then pulled with tethers 1060 in direction 1062 toward a front passage 1154. Spring ballast 1100 reduces in size between a wall 1144 surrounding front passage 1154 and the radial outward pressure caused by spring rods 1130 on wall 1144, causes expansion of wall 1144 into its expanded position.

In an exemplary embodiment, wall 1142 surrounding narrow passage 114 can be modified to enlarge narrow passage 114. Optionally, spring ballast 1100 comprises an inflatable material 1112 that inflates using, for example, hose 1020. In an exemplary embodiment, spring ballast 1100 is positioned in narrow passage 114 so that the diameter of spring rods 1130 is reduced. Spring ballast 1100 is inflated using hose 1020 to a pressure of between 34 atmospheres (optionally 3 atmospheres or less or 4 atmospheres or more), and causes expansion of wall 1142 radially outward, thereby increasing the diameter of narrow passage 114, thereby increasing blood flow.

In an exemplary embodiment, wall 1142 responds to expansion pressure. In an exemplary embodiment, if narrow passage 114 requires further expansion, spring ballast 1100 is again positioned in narrow passage 114 and inflated. In an exemplary embodiment, spring ballast 1100 is inflated to a second pressure, for example, of between 7-8 atmospheres (optionally 7 atmospheres of less or 8 atmospheres or more) to cause further expansion of wall 1142, thereby increasing the diameter of narrow passage 114.

Alternatively or additionally, wall 1142 is rigid and expansion pressure caused by inflating spring ballast 1100 causes an increase in the diameter of flow passage 114 and an outward bowing of wall 1142 to press radially outward on coronary sinus 110. In an exemplary embodiment, the apex of bowing is along wall area 1142, against coronary sinus 110 and narrow flow passage 114 is thereby widened to allow increased blood flow therethrough.

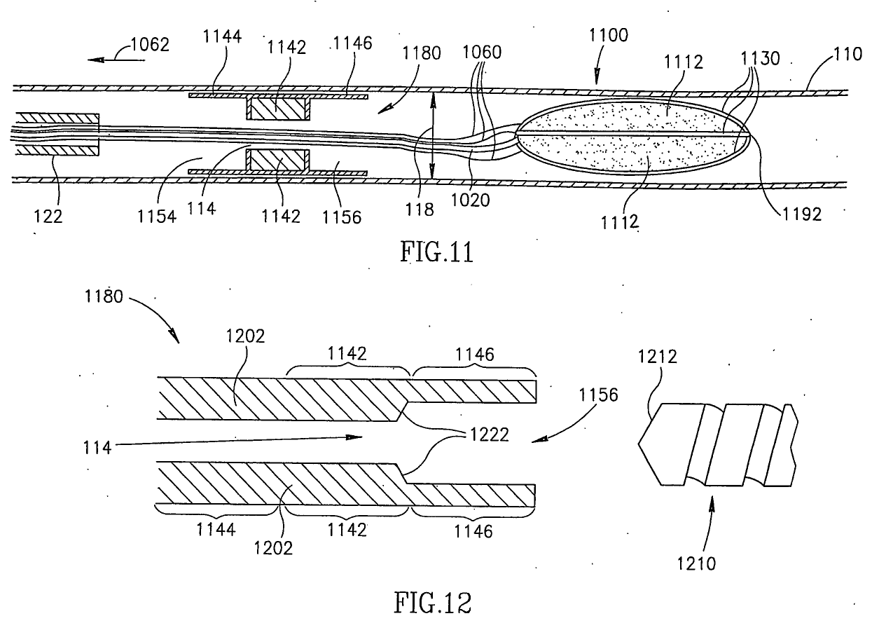

FIG. 12 shows step type flow reducing implant 1180 (FIG. 11) during manufacture, in accordance with an exemplary embodiment of the invention. In an exemplary embodiment, step type flow reducing implant 1180 comprises a tubular wall 1202 that is initially of a single thickness throughout defining narrow passage 114 its entire length. In an exemplary embodiment, a boring drill 1210 is bored into wall 1202 to create rear wall 1146 that is narrower than wall 1202 and defines rear passage 1156.

In an exemplary embodiment, boring drill 1210 is drilled into wall 1202 along front wall 1144 to create a front passage 1154 (FIG. 11) defining front wall 1144. Wall 1142, is then left in an undrilled state to define narrow passage 114.

In an exemplary embodiment, wall 1142 may be further drilled to increase the diameter of narrow passage 114. Alternatively or additionally, wall 1202 comprises a material that responds to two or more expansion pressures. In an exemplary embodiment, spring ballast 1100 is inflated to two or more inflation pressures, as described above, to provide two or more diameters of narrow passage 114.

In an exemplary embodiment, boring drill 1210 has a bevel 1212 so that in drilling wall 1142, it leaves a slanted wall 1222, allowing a specific pattern of blood flow as it passes through narrow passage 114 that promotes angiogensis and/or decreased turbulence. Alternatively or additionally, wall 1142 adjacent to narrow passage 114, can be further modified in shape, for example comprising grooves (not shown) along narrow passage 114 to further influence blood flow dynamics that promote angiogenesis.

It should be appreciated that in a slotted implant, such boring and/or forming may be performed before or after laser (or other cutting) used to form the cut-outs (e.g., as in FIG. 9).

While the invention has been described with respect to a limited number of embodiments, it will be appreciated that many variations, modifications and other applications of the invention may be made. For example, use of the flow reducing implant 700 is not restricted to application in the coronary sinus, but may be used in other veins, cavities and/or vessels related to circulation where reduction in circulation may promote angiogenesis.

A variety of values have been utilized to describe the invention including, diameters, lengths and types materials of the various flow reducing implants. Although a variety of values and/or materials have been provided, it should be understood that these could vary even further based upon a variety of engineering principles, materials, intended use and designs incorporated into the invention.

It should be appreciated that different features may be combined in different ways. In particular, not all the features shown above in a particular embodiment are necessary in every similar exemplary embodiment of the invention. Further, combinations of features from different embodiments into a single embodiment or a single feature are also considered to be within the scope of some exemplary embodiments of the invention. In addition, some of the features of the invention described herein may be adapted for use with prior art devices, in accordance with other exemplary embodiments of the invention. The particular geometric forms and measurements used to illustrate the invention should not be considered limiting the invention in its broadest aspect to only those forms. Although some limitations are described only as method or apparatus limitations, the scope of the invention also includes apparatus designed to carry out the methods and methods of using the apparatus.

Also within the scope of the invention are surgical kits, for example, kits that include sets of delivery systems and flow reducing implants. Optionally, such kits also include instructions for use. Measurements are provided to serve only as exemplary measurements for particular cases, the exact measurements applied will vary depending on the application. When used in the disclosure and/or claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. An intra-vascular balloon, comprising:
a balloon body, and
at least one springy and elongate stave attached to said balloon and conforming to a surface of said balloon, such that said stave can apply contact force to an object in contact with said balloon.

2. A balloon according to claim 1, wherein said balloon is elongate and wherein said stave is provided along a long dimension of said balloon.

3. A balloon according to claim 1, comprising a tether attached to said balloon.

4. A balloon according to claim 1, wherein said at least one stave comprise a plurality of staves arranged around said balloon.

5. A balloon according to claim 4, wherein said plurality of staves are attached to each other at their ends.

6. A balloon according to claim 5, wherein said staves modify a geometry of said balloon when not inflated.

7. A balloon according to claim 6, wherein said staves are configured to compact said balloon in a resting condition thereof.

8. A balloon according to claim 6, wherein said staves are configured to apply radially outwards pressure in a resting condition thereof.

9. A balloon according to claim 5, wherein said staves are distortable by an expansion of said balloon.

10. A balloon according to claim 1, wherein said balloon is formed of an elastic material.

11. A balloon according to claim 4, wherein said plurality of staves are configured to substantially surround said balloon when said balloon is collapsed.

12. A vascular implant, comprising:
a flexible band having a diameter suitable for implantation in a blood vessel; and
a plurality of elongate axial elements mounted on said band.

13. An implant according to claim 12, wherein said flexible band is thin.

14. An implant according to claim 12, wherein said flexible band has a thickness suitable for restricting blood flow

15. An implant according to claim 12, wherein said flexible band has a length substantially smaller than a length of said elements.

16. An implant according to claim 12, wherein said flexible band is elastic.

17. A blood flow reducing implant, comprising a body defining a flow channel having an cross-section which is progressively restricted along an axial direction, in which the smallest diameter of a cross-section is sized for passage of a guidewire and blockage of substantially all blood-flow therethrough.

18. An implant according to claim 17, wherein said cross-section is monotonicly restricted along said direction.

19. An implant according to claim 17, wherein said smallest diameter blocks over 95% of blood flow through said implant.

20. An implant according to claim 17, wherein said smallest diameter is restricted by an elastic sheath.
